(19) United States
(12) Patent Application Publication
(10) Pub. No.: US 2022/0104710 A1
(43) Pub. Date: Apr. 7, 2022

(54) BLOOD PRESSURE DEVICE

(71) Applicant: Smart Meter Corporation, Montabello, NY (US)

(72) Inventor: Benjamin Atkin, Bergenfield, NJ (US)

(73) Assignee: Smart Meter Corporation, Montabello, NY (US)

(21) Appl. No.: 17/494,137

(22) Filed: Oct. 5, 2021

Related U.S. Application Data
(60) Provisional application No. 63/088,204, filed on Oct. 6, 2020.

(51) Int. Cl. A61B 5/022; A61B 5/021; A61B 5/00

(52) U.S. Cl. CPC A61B 5/022; A61B 5/02141; A61B 5/7425; A61B 5/002; A61B 5/0004; A61B 5/0022; A61B 5/749

(57) ABSTRACT

An electronic blood pressure monitor is described. The monitor includes a blood pressure measurement unit. The unit includes a cuff fitted to a blood pressure measurement site, a pressure adjustment unit configured to adjust a pressure applied to the cuff, a pressure detecting unit configured to detect a pressure within the cuff, and a blood pressure calculation unit configured to calculate a blood pressure based on the detected pressure. The monitor also includes a memory comprising a calculation unit, a display unit configured to display the blood pressure data, a storage unit configured to store data associated with the blood pressure calculated by the calculation unit, and a read unit comprising a comparison unit. The comparison unit is configured to compare the blood pressure data from the memory with reference blood pressure data and display, via the display unit, a result of the comparison.

### CROSS-REFERENCE TO RELATED APPLICATIONS SECTION

This application is a U.S. Non-Provisional patent application that claims priority to U.S. Provisional Patent Application Ser. 63/088,204 filed on Oct. 6, 2020, the entire contents of which are hereby incorporated by reference in their entirety.

### FIELD OF THE EMBODIMENTS

The field of the invention and its embodiments relate to an improved electronic blood pressure device.

### BACKGROUND OF THE EMBODIMENTS

Hypertension is currently one of the most common diseases in the United States. In order to fight this epidemic, close monitoring and regulation of patients is strongly suggested by medical professionals. Sphygmomanometers, or blood pressure monitors, are devices used to measure blood pressure of an individual and are composed of an inflatable cuff that is configured to collapse and then release the artery under the cuff in a controlled manner. A mercury or aneroid manometer measures the pressure. Manual sphygmomanometers are used in conjunction with a stethoscope when using the auscultatory technique.

As an alternative to the traditional blood pressure monitors, digital blood pressure monitors provide convenience for the patient to monitor their blood pressure regularly. Common digital blood pressure monitors present codes in response to a blood pressure measurement taken, which are often confusing for the patient. Moreover, such digital blood pressure monitors lack the ability to store a multitude of blood pressure measurements. As such, what is needed is an accurate and improved electronic blood pressure device that avoids such pitfalls in most digital blood pressure monitors.

### Examples of Related Art Include

U.S. Pat. No. 6,699,195 B2 describes inputting time habit data representing daily time habits of a patient and timings for blood pressure measurement determined by a medical doctor into a blood pressure monitor. The predetermined timings are variably adjusted based on the daily time habit data, and results of blood pressure measurements at the specified timings are stored in a memory. A personal computer for the doctor of a medical facility receives and outputs information read from the memory of the blood pressure monitor. Accordingly, blood pressure measurements can be performed in timings adjusted in accordance with the daily habit pattern of the patient, so that more accurate measurement can be achieved.

U.S. Published Patent Application No. 2004/0176692 A1 describes a blood pressure monitor that associates information representing measuring time input from a clock with measured blood pressure values so as to store them in a memory. Blood pressure values measured in a morning time zone and blood pressure values measured in an evening time zone are grouped based on the time information associated with the blood pressure values so as to be stored in the memory. A data intra-group average calculating section calculates averages of the blood pressure values in the groups, and a risk calculating section calculates a risk value based on the calculated results of the data intra-group average calculating section.

U.S. Pat. No. 4,796,184 B2 describes a device that measures blood pressure automatically using the oscillometric technique. The device includes a pressure cuff that is automatically inflated to an initial pressure, which is calculated to be above the subject's systolic pressure. The cuff is then deflated stepwise in preset pressure increments while cuff pressure oscillations are sensed at each cuff pressure level and stored in a computer incorporated into the device. Cuff deflation is continued until the cuff pressure is below the subject's diastolic pressure, whereupon the cuff is automatically deflated. The computer then fits a calculated parabolic curve onto the greatest cuff pressure oscillation and onto a lesser oscillation on each side of the greatest oscillation. From the thusly derived curve, the computer determines the mean blood pressure, and the systolic and diastolic blood pressures.

WO 2012/078235 A1 describes a blood pressure cuff. The blood pressure cuff is defined by a first sheet and a second sheet. The first sheet has a first interior-inflatable surface attached to a second interior-inflatable surface of the second sheet to form an interior-inflatable portion between the first sheet and second sheet. The interior-inflatable portion is in communication with an opening to fluidly interconnect the interior inflatable portion with an exterior of the cuff. A textured surface is included on the second interior-inflatable surface.

Some blood pressure measurement devices are known in the art. However, their means of operation are substantially different from the present disclosure, as the other inventions fail to solve all the problems taught by the present disclosure.

### SUMMARY OF THE EMBODIMENTS

The present invention and its embodiments relate to an improved electronic blood pressure device. A first embodiment of the present invention describes an electronic blood pressure monitor. The electronic blood pressure monitor includes a blood pressure measurement unit. The blood pressure measurement unit includes numerous components, such as: a cuff fitted to a blood pressure measurement site, a pressure adjustment unit configured to adjust a pressure applied to the cuff, a pressure detecting unit configured to detect a pressure within the cuff adjusted by the pressure adjustment unit, and a calculation unit configured to calculate a blood pressure based on the detected pressure.

The electronic blood pressure monitor also includes a memory. In examples, the memory includes a calculation unit. The electronic blood pressure monitor further includes a display unit configured to display the blood pressure data, a time unit configured to indicate a time the blood pressure measurement was taken, and a storage unit configured to store data associated with the blood pressure calculated by the calculation unit in association with condition data. In some examples, the time the blood pressure measurement was taken is saved in the memory via a timestamp. The condition data indicates a condition concerning the blood pressure at the time the blood pressure measurement was taken. In examples, the calculation unit is further configured to: calculate an average of the blood pressure data stored in the memory for a time period (e.g., a day, a week, or a month).

Moreover, the electronic blood pressure monitor further includes a read unit. In some examples, the read unit includes a comparison unit. The comparison unit is configured to compare the blood pressure data from the memory with predetermined reference blood pressure data and display, via the display unit, a result of the comparison.

In additional examples, the display unit is further configured to display the blood pressure data of two or more users. Further, a user is configured to progress from the blood pressure data of a first user of the two or more users to a second user of the two or more users in response to execution of an action on a progression component of the display unit. In a further example, the display unit is configured to display wording, rather than codes, associated with the blood pressure or an error message associated with the electronic blood pressure monitor.

In some examples, a process to capture the blood pressure measurement occurs automatically and in real-time in response to a voice command from a user. In other examples, the calculation unit is configured to calculate the blood pressure automatically and in real-time in response to the voice command from a user.

A second embodiment of the present invention describes a data processing apparatus. The data processing apparatus includes a detachable memory configured to store blood pressure data calculated with a blood pressure measurement. The blood pressure data is stored in association with condition data. The data processing apparatus also includes a manipulation unit configured to manipulate the conditions and a read and display unit configured to read the blood pressure data from the detachable memory and display the read data on a display unit in response to manipulation of the manipulation unit. In some examples, the data processing apparatus further includes a communication unit configured to communicate with and/or transmit the blood pressure data to another device via Wi-Fi, Bluetooth, Bluetooth Low Energy (Bluetooth LE), or near-field communication (NFC). In further examples, the data processing apparatus is cellular-enabled.

In general, the present invention succeeds in conferring the following benefits and objectives.

The present invention provides an improved electronic blood pressure device that has an enhanced interface configured to display blood pressure results of two or more users.

The present invention provides an improved electronic blood pressure device that is voice activation enabled such that a process to capture the blood pressure measurement occurs automatically and in real-time in response to a voice command from a user.

The present invention provides an improved electronic blood pressure device that is voice activation enabled such that a calculation of the blood pressure occurs automatically and in real-time in response to a voice command from a user.

The present invention provides an improved electronic blood pressure device having a self-calibration process.

The present invention provides an improved electronic blood pressure device that is configured to display wording, rather than codes, associated with the blood pressure or an error message associated with the electronic blood pressure monitor.

The present invention provides an improved electronic blood pressure device that is configured to store, in a memory, blood pressure measurements with a timestamp to indicate a time the blood pressure measurement was taken.

The present invention provides an improved electronic blood pressure device that is configured to easily average multiple blood pressure measurements for a user.

The present invention provides a data processing apparatus that is configured to communicate with and/or transmit the blood pressure data to another device via Wi-Fi, Bluetooth, Bluetooth Low Energy (Bluetooth LE), or near-field communication (NFC).

The present invention provides a data processing apparatus that is cellular-enabled.

### BRIEF DESCRIPTION OF THE DRAWINGS

FIG. 1 depicts a block diagram of an electronic blood pressure monitor, according to at least some embodiments disclosed herein.

FIG. 2 depicts a schematic diagram of an electronic blood pressure monitor, according to at least some embodiments disclosed herein.

FIG. 3 depicts a block diagram of a method to use an electronic blood pressure monitor, according to at least some embodiments disclosed herein.

FIG. 4 depicts a schematic diagram of a data storage example, according to at least some embodiments disclosed herein.

FIG. 5-FIG. 11 depict schematic diagrams of a display portion and a main body of an electronic blood pressure monitor, according to at least some embodiments disclosed herein.

FIG. 12 depicts a block diagram of a computer, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

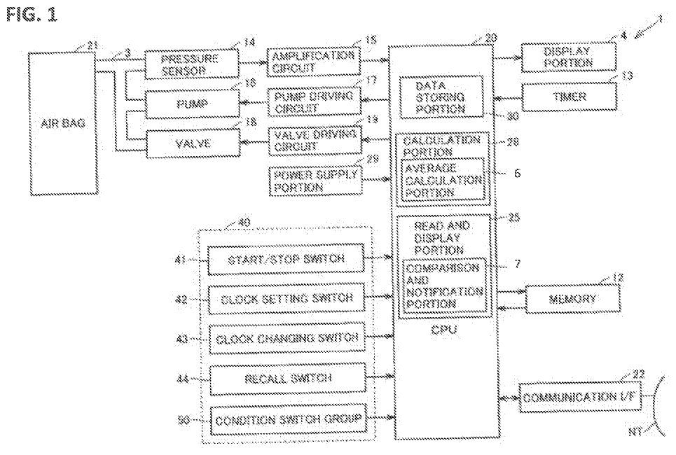

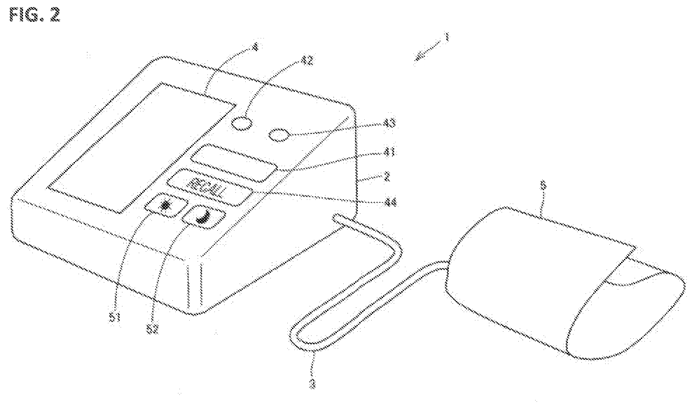

FIG. 1 and FIG. 2 depict a block diagram and a schematic diagram, respectively, of an electronic blood pressure monitor 1. The electronic blood pressure monitor 1 includes numerous components, such as a cuff 5 (of FIG. 2). The cuff 5 is fitted to a blood pressure measurement site of a user/subject and is pressurized by an air pressure and an air tube 3 connecting a main body 2 of the electronic blood pressure monitor 1 to the cuff 5. The main body 2 of the electronic blood pressure monitor 1 includes a display portion 4.

The display portion 4 of the main body 2 of the electronic blood pressure monitor 1 displays content to the user/subject. The main body 2 of the electronic blood pressure monitor 1 also includes numerous components of a manipulation portion 40 (of FIG. 1) that are manipulatable by the user/subject, such as a start/stop switch 41, a clock setting switch 42, a clock changing switch 43, a recall switch 44, a morning switch 51, an evening switch 52, and/or a condition switch group 50, among others not explicitly listed herein.

The start/stop switch 41 is manipulated to start and stop a blood pressure measurement of the user/subject. The clock setting switch 42 and the clock changing switch 43 may be manipulated to set and change a time of a timer 13 provided in the electronic blood pressure monitor 1. The recall switch 44 is manipulated to designate operations to read data regarding a blood pressure measurement result stored in a memory 12 and display the read data on the display portion 4 of the main body 2 of the electronic blood pressure monitor 1. It should be appreciated that the memory 12 stores various data described herein, including measurement data.

The morning switch 51 may be manipulated to recall stored blood pressure measurement data collected for a given user in a morning time zone. The evening switch 52 may be manipulated to recall stored blood pressure measurement data collected for a given user in the evening time zone. The condition switch group 50 includes numerous switches corresponding to respective measurement conditions that may be manipulated to designate the corresponding blood pressure measurement conditions. In some examples, the morning switch 51 and the evening switch 52 are included in the condition switch group 50.

Further, the electronic blood pressure monitor 1 includes a pressure sensor 14 that outputs a change in a pulse pressure of the user/subject at the measurement site detected via an air bag 21 contained in the cuff 5 as a pulse wave signal. The electronic blood pressure monitor 1 also includes an amplification circuit 15 that amplifies a voltage signal indicating the pressure output from the pressure sensor 14. A pump 16 and a valve 18 are configured to adjust a pressurizing (air pressure) level of the air bag 21. The electronic blood pressure monitor 1 further includes a pump driving circuit 17 and a valve driving circuit 19. The pump driving circuit 17 drives the pump 16 and the valve driving circuit 19 adjusts an opening/closing of the valve 18.

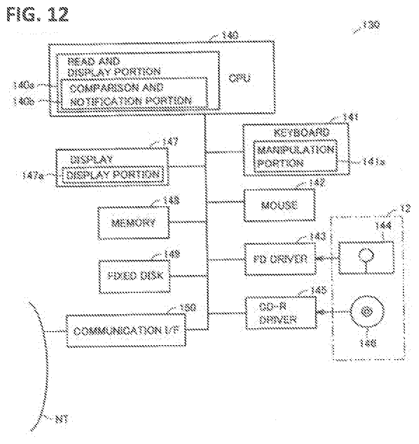

The electronic blood pressure monitor 1 additionally includes the memory 12, a timer 13 that performs time-counting operation and outputs the time data, a communication interface 22, a power supply portion 29, and a central processing unit (CPU) 20 that controls the respective portions. It should be appreciated that the communication interface 22 controls communication of the electronic blood pressure monitor 1 with an external wired or wireless communication line NT. Further, the CPU 20 communicates with an external data processing apparatus 130 (of FIG. 12) via the communication interface 22 and the communication line NT. For example, the CPU 20 may transmit data of blood pressure measurement results read from the memory 12 to the data processing apparatus 130 via the communication interface 22 and the communication line NT.

The CPU 20 has a memory (not shown) for temporarily storing data, a read and display portion 25, a calculation portion 28 for calculating blood pressure and pulsation, and a data storing portion 30 having a function of storing blood pressure measurement data in the memory 12. Moreover, the CPU 20 has a function for processing the blood pressure measurement data. The read and display portion 25 controls the display on the display portion 4. The functions of read and display portion 25, the calculation portion 28, and the data storing portion 30 are realized as the CPU 20 reads and executes a prescribed program from the memory 12.

The calculation portion 28 includes an average calculation portion 6 that calculates an average of the blood pressure measurement data. In some examples, the memory 12 may be formed of a partial memory storing various programs and data controlling the operation of the electronic blood pressure monitor 1 and a partial memory storing the blood pressure measurement data, and the partial memory storing the blood pressure measurement data may be provided to the electronic blood pressure monitor 1 in a detachable manner.

As shown in FIG. 1, the air bag 21 is connected to the pressure sensor 14, the pump 16, and the valve 18 via the air tube 3. The power supply portion 29 is configured to supply power for driving the respective portions and is formed of a battery or a commercial power source. The calculation portion 28 is configured to calculate a blood pressure value and a pulse rate based on a pulse signal input from the amplification circuit 15.

During blood pressure measurement, the calculation portion 28 is configured to convert the pulse signal output from the amplification circuit 15 to digital data. The calculation portion 28 is then configured to apply an algorithm to the data to calculate a systolic blood pressure, a diastolic blood pressure, and a pulse rate.

As described herein, the “systolic blood pressure” measures the force of blood against an individual's artery walls while the individual's ventricles squeeze and push blood out to the rest of the individual's body. For a typical adult, the systolic blood pressure is normally less than 140 mmHg.

As described herein, the “diastolic blood pressure” measures the force of blood against the individual's artery walls as the individual's heart relaxes and the ventricles are allowed to refill with blood. Diastole—the period of time when the individual's heart relaxes between beats—is also the time that the individual's coronary artery is able to supply blood to the individual's heart. For a healthy adult, the diastolic blood pressure is normally less than 90 mm Hg.

As defined herein, the “pulse rate” is the number of heartbeats of the individual per minute. The resting pulse rate for an average adult is between 60 and 80 beats per minute.

It should be appreciated that the electronic blood pressure monitor 1 comprises a self-calibration test. Accuracy of blood pressure measurements is dependent upon the “exhaust velocity”, or deflation rate, of the cuff 5. The electronic blood pressure monitor 1 is configured to operate with a deflation rate of 2 to 3 mm Hg per step, as recommended by the American Heart Association (AHA). See, Liz Smith, “New AHA Recommendations for Blood Pressure Measurement,” Am Fam Physician, 2005, 72(7), Pages 1391-1398, the contents of which are hereby incorporated by reference in its entirety.

The electronic blood pressure monitor 1 comprises an analysis component (not shown) that is configured to, automatically and in real-time, detect the deflation rate and compare the deflation rate to the predetermined deflation rate of 2 to 3 mm Hg per step. In response to a determination that the deflation rate is greater than the predetermined deflation rate of 2 to 3 mm Hg per step, the electronic blood pressure monitor 1 is configured to, automatically and in real-time, decrease the deflation rate to the predetermined deflation rate. In response to a determination that the deflation rate is less than the predetermined deflation rate of 2 to 3 mm Hg per step, the electronic blood pressure monitor 1 is configured to, automatically and in real-time, increase the deflation rate to meet the predetermined deflation rate of 2 to 3 mm Hg per step. It should be appreciated that the specifics of the self-calibration test are provided for illustrative purposes only and other self-calibration tests are contemplated by Applicant's disclosure.

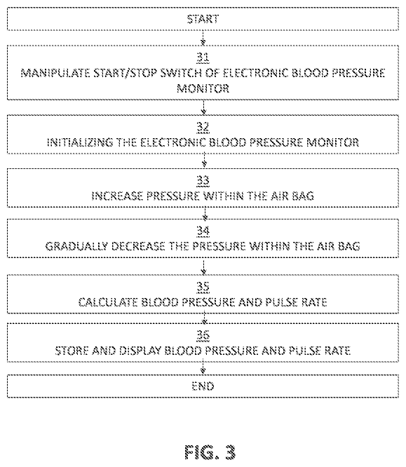

FIG. 3 depicts a block diagram of a method to use the electronic blood pressure monitor 1. The method of FIG. 3 includes numerous process steps. It should be appreciated that the process steps of FIG. 3 are pre-stored in the memory 12 as a program and are read and executed by the CPU 20.

A process step 31 begins the method of FIG. 3 and includes the user/subject winding the cuff 5 around the measurement site (e.g., the upper arm, the wrist, and/or the finger of the user/subject) and manipulating the start/stop switch 41 of electronic blood pressure monitor 1. In response to such manipulation, the manipulation signal is applied to the CPU 20. The CPU 20, in response to the applied manipulation signal, controls the power supply portion 29 to start power supply to the respective portions.

A process step 32 follows the process step 31 and includes initializing the electronic blood pressure monitor 1. During this process step, the CPU 20 controls portions of FIG. 1 to evacuate the air within air bag 21 such that the output level of pressure sensor 14 is 0 mmHg. Next, a process step 33 follows the process step 32 and includes the CPU 20 controlling certain portions of FIG. 1 to increase the pressure within air bag 21 to the systolic blood pressure of the subject +40 mmHg.

A process step 34 follows the process step 33 and includes the CPU 20 gradually decreasing the pressure within air bag 21. During this process, the pressure within air bag 21 is detected by the pressure sensor 14. A process step 35 follows the process step 34 and includes the calculation portion 28 of the CPU 20 calculating the systolic blood pressure value, the diastolic blood pressure value, and the pulse rate of the user/subject based on the detected pressure.

It should be appreciated that the method of FIG. 3 may measure the systolic blood pressure value, the diastolic blood pressure value, and/or the pulse rate of the user/subject via a pressure-increasing process. In further examples, the systolic blood pressure value, the diastolic blood pressure value, and the pulse rate of the user/subject may be calculated as an average based on numerous factors and conditions. For example, the systolic blood pressure value for the user/subject may be calculated as an average for a week's time. In another example, the diastolic blood pressure value for the user/subject may be calculated as an average for every morning (e.g., between 4:00 AM-10:00 AM) for a month's time. The CPU 20 then temporarily stores the systolic blood pressure value, the diastolic blood pressure value, and the pulse rate of the user/subject in an internal memory of the CPU 20.

A process step 36 follows the process step 35 and includes displaying, via the read and display portion 25, the systolic blood pressure value, the diastolic blood pressure value, and/or the pulse rate of the user/subject on display portion 4. The data storing portion 30 of the CPU 20 is then configured to read the systolic blood pressure value, the diastolic blood pressure value, and the pulse rate of the user/subject temporarily stored in the internal memory of the CPU 20 and store the systolic blood pressure value, the diastolic blood pressure value, and the pulse rate of the user/subject in the memory 12 in association with time data counted by the timer 13 as the measurement time data.

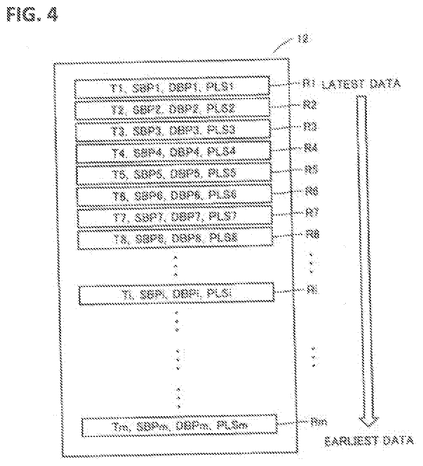

It should be appreciated that measurement results (e.g., the systolic blood pressure value, the diastolic blood pressure value, and/or the pulse rate of the user/subject) may be stored in the memory 12 as shown in FIG. 4. FIG. 4 depicts a schematic diagram of one such data storage example, which is provided for illustrative purposes only. It should be appreciated that other manners of data storage are contemplated by Applicant's disclosure.

In a further example, the electronic blood pressure monitor 1 may comprise a voice activation component, engine, application, or program, such that the method of FIG. 3 may begin upon detection and recognition of a predetermined voice command stored in the memory 12.

In FIG. 4, the measurement results are stored in the memory 12 in units of records Ri (i=1, 2, . . . , m). For example, a Record Ri includes measurement time data Ti, systolic blood pressure data SBPi, diastolic blood pressure data DBPi, and pulse rate data PLSi. For the measurement time data Ti, the CPU 20 receives data of the start or end time when the blood pressure was measured, as determined by the timer 13, and converts the data to measurement time data Ti (including year, month, day, hour, and/or minute), which is stored in the Record Ri. As such, the measurement time data Ti indicates a measurement condition concerning the corresponding systolic blood pressure data SBPi and diastolic blood pressure data DBPi. In some examples, the measurement time data Ti of the Record Ri may be a timestamp.

As depicted in FIG. 4, the Records Ri are stored in the memory 12 in a chronological sequence of measurement times, from the data measured most recently to the data measured most previously. It should be appreciated that in other examples, the Records Ri may be stored in the memory 12 in another chronological sequence of measurement times, from the data measured most previously to the data measured most recently.

If the user/subject wishes to recall the blood pressure measurement data stored in the memory 12, the user/subject may first manipulate the recall switch 44. In response, the read and display portion 25 of the CPU 20 starts reading the Record Ri stored most recently from the memory 12 and displays the measurement result on the display portion 4. This process is carried out every time the user/subject manipulates the recall switch 44 to read the Records Ri from the memory 12 from the latest (most recent) to the earliest (least recent). It should be appreciated that in some examples, the Record Ri may be recalled from the memory 12 in the opposing order, from the earliest (least recent) to the latest (most recent).

In another example, the user/subject may manipulate the morning switch 51. In response, the read and display portion 25 of the CPU 20 reads the Record Ri from the memory 12 that was stored most recently and determines whether the time data Ti of the read Record Ri indicates any time from 4:00 AM to 10:00 AM. In response to a determination that the time data Ti of the read Record Ri fails to fall within the time frame from 4:00 AM to 10:00 AM, the read and display portion 25 of the CPU 20 reads the next latest Record Ri from the memory 12 and then assesses whether the time data Ti of the read Record Ri indicates any time from 4:00 AM to 10:00 AM. It should be appreciated that this process assumes that the data indicating the time from 4:00 AM to 10:00 AM is stored in advance in memory 12 or incorporated in the logic of the program, or input by the user/subject.

If the read and display portion 25 determines that the time data Ti of the Record Ri indicates any time from 4:00 AM to 10:00 AM, the read and display portion 25 displays the measurement result on the display portion 4 based on the content of the relevant Record Ri. This process is repeated every time the morning switch 51 is manipulated. It should be appreciated that the same process steps may occur for the evening switch 52 for an evening time frame, such as 5:00 PM to 9:00 PM.

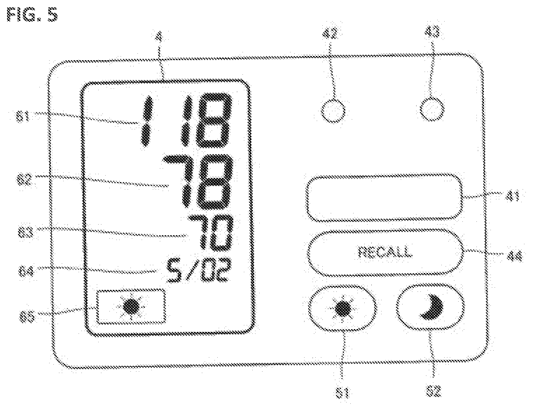

FIG. 5-FIG. 8 depict schematic diagrams of the display portion 4 and the main body 2 of the electronic blood pressure monitor 1. As described, the main body 2 of the electronic blood pressure monitor 1 may include: the start/stop switch 41, the clock setting switch 42, the clock changing switch 43, the recall switch 44, the morning switch 51, and/or the evening switch 52, among other components not explicitly listed herein.

The display portion 4 of the electronic blood pressure monitor 1 may display the Record Ri associated with measurement data, as explained previously. For example, as shown in FIG. 5, the display portion 4 may display systolic blood pressure data 61 based on systolic blood pressure data SBPi, diastolic blood pressure data 62 based on diastolic blood pressure data DBPi, pulse rate data 63 based on pulse rate data PLSi, and time data 64 based on measurement time data Ti. The time data 64 may display the month, the date, the hour, and/or the minute.

Also shown in FIG. 5, the display portion 4 may display a mark 65. The mark 65 may coincide with the time frame associated with the Record Ri. For example, and as depicted in FIG. 5, if the Record Ri is associated with the morning time frame, the mark 65 may be associated with a morning symbol, such as a sun. In another example, if the Record Ri is associated with the evening time frame, the mark 65 may be associated with an evening symbol, such as a moon.

In an alternative embodiment, the systolic blood pressure data 61, the diastolic blood pressure data 62, the pulse rate data 63, and the time data 64 may be displayed in order from the earliest to the latest. In further examples, the systolic blood pressure data 61, the diastolic blood pressure data 62, the pulse rate data 63, and the time data 64 may be displayed in order from the latest to the earliest. In a further example, the voice activation component, engine, application, or program may display the measurement data in response to detection and recognition of a predetermined voice command stored in the memory 12.

In additional examples, the display portion 4 of the electronic blood pressure monitor 1 may display the Record Ri associated with the most recent measurement data for the user/subject, along with the most recent 100 Records Ri.

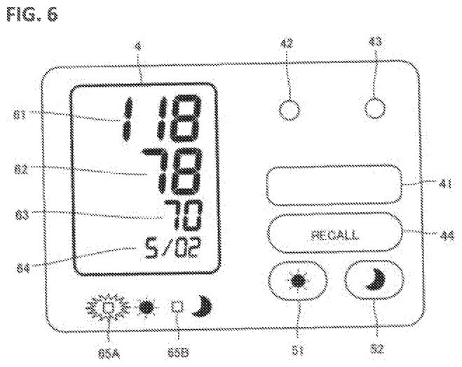

In a further embodiment, and as depicted in FIG. 6, the mark 65 may be replaced with a first light emitting diode (LED) 65A and a second LED 65B. In an example, the first LED may correlate to a morning time zone/frame and the second LED 65B may correlate to an evening time zone/frame. In this example, the read and display portion 25 may be configured to turn the first LED 65A and/or the second 65B on in accordance with the measurement condition (e.g., the morning or evening time zone/frame) of the blood pressure measurement data being displayed. In a further example, the first LED 65A may be on and the second LED 65B may be off, in response to the manipulation of morning switch 51, to show that the measurement data being displayed is associated with the morning time zone/frame.

In another embodiment, the measurement data associated with the morning time frame may be recalled sequentially every time the morning switch 51 is manipulated by the user/subject. Alternatively, the measurement data associated with the evening time frame may be recalled sequentially every time the evening switch 52 is manipulated by the user/subject.

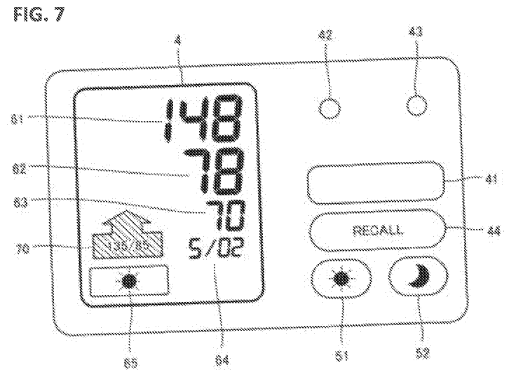

In some examples and in accordance with FIG. 7, a comparison and notification portion 7 of the read and display portion 25 of FIG. 1 may compare the systolic blood pressure data 61 to a reference value and the diastolic blood pressure data 62 to a reference value. Moreover, the reference values may also be pre-stored in an internal memory in the CPU 20.

A result of the comparison may be displayed via a notification mark 70 (of FIG. 7). In some examples, the notification mark 70 may be displayed in addition to the display data, as depicted in FIG. 7. If the comparison and notification portion 7 determines that the systolic blood pressure data 61 is greater than or equal to the reference value and/or the diastolic blood pressure data 62 is greater than or equal to the reference value, the notification mark 70 may be displayed. As depicted in FIG. 7, the notification mark 70 may take the shape of an upwards arrow to indicate that one or more of the systolic blood pressure data 61 and the diastolic blood pressure data 62 are higher than expected, based on the reference values. In further examples, the notification mark 70 may be lighted while displayed. In other examples, the notification mark 70 will be displayed via one or more blinking lights. In additional examples, the electronic blood pressure monitor 1 may vibrate and/or the electronic blood pressure monitor 1 may produce one or more sounds for notification to the user/subject if the systolic blood pressure data 61 and/or the diastolic blood pressure data 62 are higher than expected.

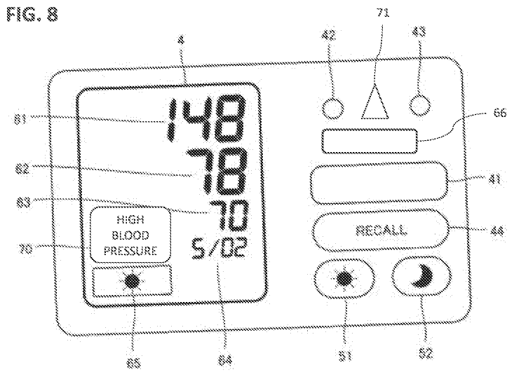

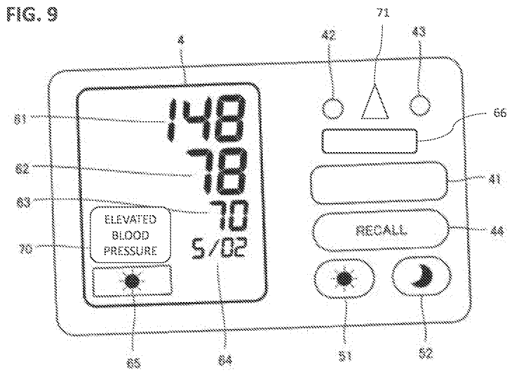

As shown in FIG. 8 and FIG. 9, the notification mark 70 need not be pictorial and may include wording to alert the user/subject of the user's/subject's blood pressure. For example, as shown in FIG. 8, the notification mark 70 may display the wording, “High Blood Pressure,” to indicate that the user/subject's, assuming the user/subject is an average adult, has a systolic blood pressure between 130-139 mmHg and/or has a diastolic blood pressure between 80-89 mmHg. As shown in FIG. 9, the notification mark 70 may display the wording, “Elevated Blood Pressure,” to indicate that the user/subject's, assuming the user/subject is an average adult, has a systolic blood pressure between 120-129 mmHg and/or has a diastolic blood pressure less than 80 mmHg. Other appropriate wording may include, but is not limited to, “Normal Blood Pressure,” “Hypertensive Crisis—Seek Emergency Care,” “High Blood Pressure—Stage 1,” and/or “High Blood Pressure—Stage 2.”

In other examples, the notification mark 70 may display wording associated with error messages of the electronic blood pressure monitor 1. The display of wording, rather than codes, for error messages and the status of the blood pressure of the user/subject is provided for user convenience and ease of understanding.

It should further be appreciated that in some examples, the measurement data may be recalled after the user/subject gets up, before the user/subject goes to bed, before the user/subject eats a meal, after the user/subject eats the meal, before the user/subject exercises, before the user/subject takes a medication, after the user/subject takes the medication, and/or after the user/subject exercises, among others. The user/subject may manipulate a switch in the condition switch group 50 corresponding to the desired measurement condition (e.g., before the user/subject exercises).

An example storage associated with these conditions may include grouping the Records Ri of measurement result data based on the conditions designated at the time the blood pressure measurement was taken and storing them in areas provided in advance in the memory 12 for the respective groups. For example, the measurement results may be stored in units of the Records Ri. Each of the Records Ri includes the measurement time data Ti, the systolic blood pressure data SBPi, the diastolic blood pressure data DBPi, and the pulse rate data PLSi. It should be appreciated that this example is provided for illustrative purposes only and other examples are contemplated. In some examples, the Records Ri are stored in order from the latest to the earliest. In other examples, the measurement data may be stored in pairs in the memory 12.

In an example, the electronic blood pressure monitor 1 may store measurement data for two or more users. In some examples, a quantity of the two or more users is four. However, it should be appreciated that the quantity of the two or more users is not limited to any particular quantity. In an example, a user or a third-party, such as a parent, may wish to view the measurement data for two or more children. In a further example, a doctor may wish to view the measurement data for the two or more patients.

Each of the two or more users who have blood pressure measurements taken by the electronic blood pressure monitor 1 may be associated with a unique identifier stored in the memory 12. Such unique identifier is also associated with the measurement data for that user. As shown in FIG. 8, the main body 2 of the electronic blood pressure monitor 1 includes a user component 66.

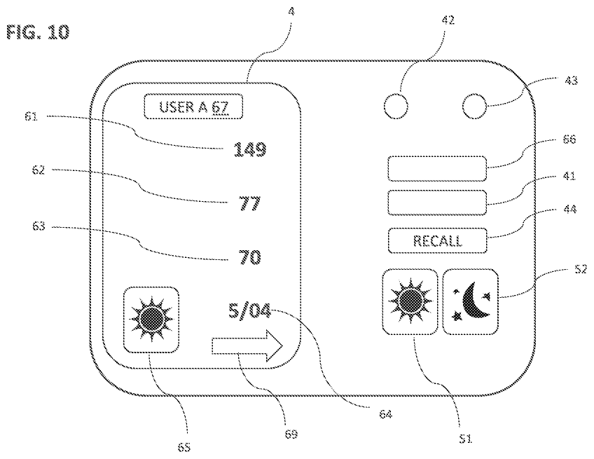

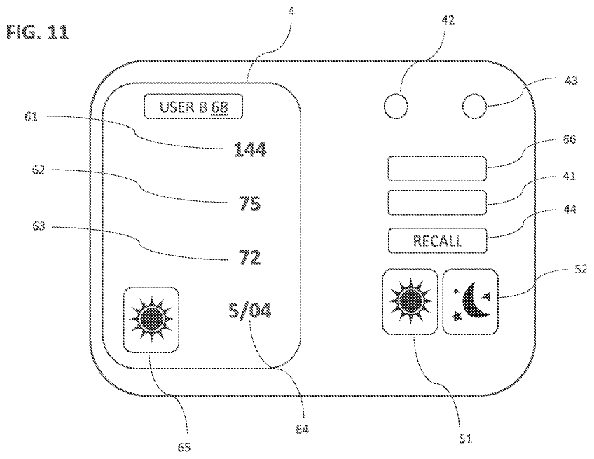

The user/third-party may interact with the user component 66 to display the Record Ri associated with measurement data for a user of the two or more users. As shown in FIG. 10, the user of the two or more users associated with the Record Ri may be a user A 67. If the user/third-party wishes to view the Record Ri associated with measurement data for another user of the two or more users, such as a user B 68 (of FIG. 11), the user/third-party may exert an action on a progression component 69 (of FIG. 10) to view the measurement data associated with the second or next user of the two or more users. In another example, the user/third-party may exert a swipe action on the display portion 4 to view the measurement data associated with the second or next user of the two or more users.

FIG. 12 depicts a block diagram of a computer, according to at least some embodiments disclosed herein. Referring to FIG. 12, the data processing apparatus 130 includes a display 147. The display 147 may be a liquid crystal display or a cathode ray tube, among other examples not explicitly disclosed herein. The data processing apparatus 130 also includes other components, such as: a keyboard 141 and a mouse 142, a CPU 140 for centrally controlling the data processing apparatus 130, a memory 148 configured to include Read-Only Memory (ROM) or Random Access Memory (RAM), a fixed disk 149, a Flexible Disk (FD) driver 143 mounted detachably with the FD 144 and accessing the mounted FD 144, a Compact Disc Recordable (CD-R) driver 145 mounted detachably with the CD-R 146 and accessing the mounted CD-R 146, and a communication interface 150 for communicably connecting data processing apparatus 130 with a communication line NT. The respective portions are connected via a bus in a communicable manner. The detachable recording medium is not limited to the FD 144 or the CD-R 146 and may include others not explicitly listed herein.

The recalling function of the measurement data described herein is realized by a program. The program is stored in a computer-readable recording medium in FIG. 12. The recording medium may be the memory 148, the FD 144, or the CD-R 146. The program recorded on the recording medium may be read and executed by the CPU 140. Alternatively, the program recorded on the recording medium may be read and loaded to a prescribed program memory area (e.g., prescribed area in the memory 148) in FIG. 12, and then read from the relevant area and executed by the CPU 140. In another example, the program may be downloaded via the communication interface 150 and the communication line NT to the memory 148.

The CPU 140 includes a read and display portion 140a. The read and display portion 140a includes a comparison and notification portion 140b. The read and display portion 140a and the comparison and notification portion 140b have functions equivalent to those of the read and display portion 25 and the comparison and notification portion 7, respectively. The functions of read and display portion 104a and the comparison and notification portion 140b are realized as the CPU 140 reads the program from the memory and executes the same.

The keyboard 141 includes a manipulation portion 141a. The manipulation portion 141a includes the condition switch group 50 and the recall switch 44. The display 147 includes a display portion 147a. The display portion 147a has functions equivalent to that of the display portion 4.

It is assumed that memory 12 storing the measured blood pressure data in each of the examples may be mounted to the electronic blood pressure monitor 1 in a detachable manner. In such a case, the memory 12 is mounted to the data processing apparatus 130 in FIG. 12 as the FD 144 or the CD-R 146. As such, the blood pressure measurement data associated with conditions in the above embodiments are supplied to the data processing apparatus 130.

Alternatively, the data processing apparatus 130 may receive the blood pressure measurement data associated with the conditions, which are read from the memory 12 and transmitted by the electronic blood pressure monitor 1, via communication line NT and the communication interface 150, and may store the received data in the FD 144 or the CD-R 146. As such, the blood pressure measurement data associated with conditions are supplied to the data processing apparatus 130. Alternatively, in the case where the blood pressure data and the condition data are transmitted separately, the received blood pressure data may be stored in the FD 144 or the CD-R 146 in association with the received condition data.

When the blood pressure measurement data associated with conditions are supplied, the user/subject can manipulate the recall switch 44 and the condition switch group 50 in the manipulation portion 141a in a similar manner as described to display the blood pressure measurement data associated with a desired condition by the display portion 147a of the display 147. As such, the user/subject can recall his/her blood pressure measurement data by manipulating the data processing apparatus 130 even if he/she is away from home.

If the user/subject wishes to view average data of the last three Records Ri's, the user/subject may select the average component/button 71 of FIG. 8 and FIG. 9. In response to such selection, the average data of the last three Records Ri's may be displayed to the user/subject on the display portion 4. It should be appreciated that the quantity of three is for illustrative purposes only and other quantities are contemplated by Applicant's disclosure.

In some examples, the data processing apparatus 130 of FIG. 12 may communicate and/or transmit measurement results to another computing device (not shown), such as a smartphone, a laptop computer, a tablet, or another suitable computing device, via Wi-Fi, Bluetooth, Bluetooth Low Energy (Bluetooth LE), and/or near-field communication (NFC).

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, Bluetooth LE, and IEEE 802.11.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

As described herein, “NFC” is a set of communication protocols for communication between two electronic devices over a distance of 4 cm or less. NFC devices can act as electronic identity documents and keycards and may be used in contactless payment systems and allow mobile payment replacing or supplementing systems such as credit cards and electronic ticket smart cards. NFC can be used for sharing small files such as contacts, and bootstrapping fast connections to share larger media such as photos, videos, and other files.

Moreover, in some examples, the data processing apparatus 130 may be cellular enabled and, in some examples, may incorporate SMC cellular patented technology, among other technologies not explicitly listed herein.

Specifically, in other examples, the data processing apparatus 130 of FIG. 12 may comprise a cellular modem (not shown) to communicate and/or transmit measurement results to another computing device (not shown), such as a smartphone, a laptop computer, a tablet, or another suitable computing device. It should be appreciated that, as described herein, the cellular modem is a device that adds cellular connectivity to laptops, desktop computers, tablets, and other similar devices. Furthermore, it should be appreciated that the cellular modem (not shown) replaces the existing BLE module in the Bluetooth devices described herein.

In examples, the cellular modem may be embedded within the data processing apparatus 130 or may be a standalone device that is connected to the data processing apparatus 130 through various means, including, but not limited to, a USB connection. Examples of cellular modems include, but are not limited to, AT&T Momentum, Verizon 551 L, USB cellular modems and motherboard mounted cellular chipsets manufactured by Novatel Wireless, Sierra Wireless, Huawei, and the like. In other examples, the cellular modem may operate by switching between cellular and satellite communications.

Furthermore, the cellular modem may be configured to automatically connect to a slower network when the faster network is not available. The cellular modem may also monitor the reliability of all available connections. The reliability of a network can be determined from information collected by the cellular modem, which includes, but is not limited to, signal strength, quality, availability, packet loss, retransmits, packet latency, throughput speed, and other cell tower signaling quality factors. The cellular modem may then compare this information in various forms to a reliability threshold in order to determine whether or not to maintain or terminate a connection to a cellular network. The reliability threshold is often automatically set by the cellular carrier, or may be manually set by the user of the data processing apparatus 130.

Further, it should be appreciated that the cellular modem is also configured to establish a connection with cellular networks in which the cellular modem is located. The cellular modem is configured to monitor and detect all cellular networks as the cellular modem moves from one network coverage area to another network coverage area via a vehicle in which it is contained. The cellular modem can detect when a connection to a particular network is made, whether it is a 3G, 4G, or 5G network, as well as which cellular network provider (e.g., Verizon, T-Mobile, etc.) it has connected to.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. An electronic blood pressure monitor comprising:
a blood pressure measurement unit comprising:
a cuff fitted to a blood pressure measurement site;
a pressure adjustment unit configured to adjust a pressure applied to the cuff;
a pressure detecting unit configured to detect a pressure within the cuff adjusted by the pressure adjustment unit; and
a blood pressure calculation unit configured to calculate a blood pressure based on the detected pressure by the pressure detecting unit;
a memory comprising a calculation unit;
a display unit configured to display the blood pressure data;
a time unit configured to detect a time of the blood pressure measurement;
a storage unit configured to store data associated with the blood pressure calculated by the calculation unit in association with condition data, the condition data indicating a condition concerning the blood pressure at the time of the blood pressure measurement; and
a read unit comprising a comparison unit, the comparison unit being configured to compare the blood pressure data from the memory with reference blood pressure data and display, via the display unit, a result of the comparison.

2. The electronic blood pressure monitor of claim 1, wherein the calculation unit is further configured to:
calculate an average of the blood pressure data for a user stored in the memory for a time period selected from the group consisting of: a day, a week, and a month.

3. The electronic blood pressure monitor of claim 1, wherein the display unit is further configured to:
display the blood pressure data of two or more users.

4. The electronic blood pressure monitor of claim 3, wherein a user is configured to progress from the blood pressure data of a first user of the two or more users to the blood pressure data of a second user of the two or more users in response to execution of an action on a progression component of the display unit.

5. The electronic blood pressure monitor of claim 1, wherein the time of the blood pressure measurement is saved in the memory via a timestamp.

6. The electronic blood pressure monitor of claim 1, wherein a process to capture the blood pressure measurement occurs automatically and in real-time in response to a voice command from a user.

7. The electronic blood pressure monitor of claim 1, wherein the calculation unit is configured to calculate the blood pressure automatically and in real-time in response to a voice command from a user.

8. The electronic blood pressure monitor of claim 1, wherein the display unit is configured to display wording associated with the blood pressure or an error message associated with the electronic blood pressure monitor.

9. A data processing apparatus comprising:
a detachable memory comprising blood pressure data calculated with a blood pressure measurement stored in association with condition data, wherein the condition data indicates a condition concerning the blood pressure data;
a manipulation unit configured to manipulate a condition; and
a read and display unit configured to read the blood pressure data from the detachable memory and display the read data on a display unit in response to a manipulation of the manipulation unit.

10. The data processing apparatus of claim 9, further comprising:
a communication unit configured to communicate with and/or transmit the blood pressure data to another device via Wi-Fi, Bluetooth, Bluetooth Low Energy (Bluetooth LE), or near-field communication (NFC).

11. The data processing apparatus of claim 9, wherein the data processing apparatus is cellular-enabled.
